(19) United States
(12) Patent Application Publication
(10) Pub. No.: US 2019/0033275 A1
(43) Pub. Date: Jan. 31, 2019

(54) TEMPERATURE VARIATION FOR SENSOR ARRAY BASED DETECTION TECHNOLOGY

(71) Applicant: Pulmostics Limited, Dublin (IE)

(72) Inventor: Yin Sun, Bridgewater, NJ (US)

(21) Appl. No.: 16/042,553

(22) Filed: Jul. 23, 2018

Related U.S. Application Data
(60) Provisional application No. 62/536,883, filed on Jul. 25, 2017.

(51) Int. Cl. G01N 33/00

(52) U.S. Cl. CPC G01N 33/0016; G01N 33/0013; G01N 33/0073; G01N 33/0065; G01N 33/0031

(57) ABSTRACT

A method for identification of a vapor sample or chemicals in a vapor sample includes introducing a vapor sample to a sensor array including a plurality of sensors, adjusting a temperature of one or more of the plurality of sensors between at least two temperature levels, and identifying the vapor sample or one or more chemicals in the vapor sample based on a plurality of response patterns of the sensor array, each of the response patterns being a collection of responses of the plurality of sensors to the vapor sample with the one or more sensors being at a different temperature level from among the at least two temperature levels.

### CROSS-REFERENCE TO RELATED APPLICATIONS

This application relates to and claims the benefit of U.S. Provisional Application No. 62/536,883 filed Jul. 25, 2017 and entitled “TEMPERATURE VARIATION FOR SENSOR ARRAY BASED DETECTION TECHNOLOGY,” the entire contents of which is expressly incorporated herein by reference.

### STATEMENT RE: FEDERALLY SPONSORED RESEARCH/DEVELOPMENT

Not Applicable

### BACKGROUND

### 1. Technical Field

The present disclosure relates generally to identification of chemicals in a sample and, more particularly, to identification of chemicals through the use of a sensor array including a plurality of sensors.

### 2. Related Art

A sensor array, sometimes referred to as an electronic nose or eNose, uses multiple sensors to classify substances based on the response pattern of the sensors. The sensors of a sensor array, which may comprise small silicon chips with electrodes, may be coated with sensory material coatings, such as polymers, nanotubes with specific function groups, nanofibers with specific function groups, or other materials that selectively respond to a certain chemical or chemicals in a sample and produce detectable signals. The selective reactions may be due to the specific reactive sites on the sensory materials that have different reaction affinity (e.g. adsorption, dissolution, or other chemical reaction affinity) to different chemicals. Depending on the types of sensors used, certain properties, such as mass, reflection rate, temperature, or the resistance of the sensory materials will be different before and after the adsorption or other reactions. By detecting the differences, establishing the response pattern of all the sensors of the sensor array, and comparing the results with a library established by training known samples or through machine learning processes, the sample or chemicals in the sample might be identified/classified or a change in chemical properties of the sample may be observed. Since the sensory materials will react to different chemicals differently, the sensor array detectors can be used to identify individual chemicals or classify mixed samples. However, it can be difficult to achieve high accuracy, especially when the sample is a complex mixture of multiple chemicals and/or when the sample includes a significant high concentration of water.

### BRIEF SUMMARY

The present disclosure contemplates various systems, methods, and apparatuses for overcoming the above drawbacks accompanying the related art. One aspect of the embodiments of the disclosure is a method for identification of a vapor sample or chemicals in a vapor sample. The method may include introducing a vapor sample to a sensor array including a plurality of sensors, adjusting a temperature of one or more of the plurality of sensors between at least two temperature levels, and identifying the vapor sample or one or more chemicals in the vapor sample based on a plurality of response patterns of the sensor array, each of the response patterns being a collection of responses of the plurality of sensors to the vapor sample with the one or more sensors being at a different temperature level from among the at least two temperature levels.

The adjusting may include continuously ramping the temperature at one or more predetermined rates over a range of temperature levels including the at least two temperature levels. The method may include receiving a temperature profile defining a varying temperature level as a function of time. The continuously ramping the temperature may be performed according to the temperature profile.

The adjusting may include holding the temperature at each of the at least two temperature levels until the responses of the one or more sensors at that temperature level reach equilibrium. The method may include receiving a temperature profile defining a set of discrete temperature levels. The holding the temperature at each of the at least two temperature levels may be performed according to the temperature profile.

The response of each of the plurality of sensors to the vapor sample may quantify a degree of adsorption of the vapor sample to the sensor. The adjusting may include initially holding the temperature at a temperature level associated with a high degree of adsorption until the responses of the one or more sensors at that temperature level reach equilibrium and subsequently adjusting the temperature in a direction that reduces the degree of adsorption. The plurality of response patterns may be arranged as a desorption profile beginning with a response pattern that is a collection of responses of the plurality of sensors to the vapor sample with the one or more sensors being at the temperature level associated with the high degree of adsorption. The adjusting may include initially holding the temperature at a temperature level associated with a low degree of adsorption until the responses of the one or more sensors at that temperature level reach equilibrium and subsequently adjusting the temperature in a direction that increases the degree of adsorption. The plurality of response patterns may be arranged as an adsorption profile beginning with a response pattern that is a collection of responses of the plurality of sensors to the vapor sample with the one or more sensors being at the temperature level associated with the low degree of adsorption.

The identifying may include searching a sensor response library for a match between each of the plurality of response patterns and one or more chemicals in the sensor response library, which may be established by training sensors with known samples using machine learning, deep learning, or other artificial intelligence methods. The sensor response library may store known response patterns in association with chemicals or combinations of chemicals. Individual components of the known response patterns may be stored in the sensor response library in association with individual sensors. The known response patterns may be stored in the sensor response library in association with the plurality of sensors of the sensor array. Individual components of the known response patterns may be stored in the sensor response library in association with individual sensors from among the plurality of sensors of the sensor array. The known response patterns may be stored in the sensor library in association with temperature levels at which the known response patterns were determined. The known response patterns may be stored in the sensor library in association with temperature profiles specifying how temperature was controlled during the determination of the known response patterns, each of the temperature profiles defining a varying temperature level as a function of time or a set of discrete temperature levels.

Each of the plurality of sensors may be of a type selected from the group consisting of: surface acoustic wave (SAW), chemoresistant, fluorescent, and metal oxide.

The plurality of sensors may include sensors of two or more types selected from the group consisting of: surface acoustic wave (SAW), chemoresistant, fluorescent, and metal oxide.

At least two of the plurality of sensors may be coated with different sensory material coatings that produce different sensor responses to the vapor sample.

Another aspect of the embodiments of the disclosure is a system for identification of a vapor sample or chemicals in a vapor sample. The system may include a sensor array including a plurality of sensors, a temperature controller that adjusts a temperature of one or more of the plurality of sensors between at least two temperature levels, and a chemical identifier that identifies a vapor sample introduced to the sensor array or one or more chemicals in a vapor sample introduced to the sensor array based on a plurality of response patterns of the sensor array, each of the response patterns being a collection of responses of the plurality of sensors to the vapor sample with the one or more sensors being at a different temperature level from among the at least two temperature levels.

Another aspect of the embodiments of the disclosure is a non-transitory program storage medium on which are stored instructions executable by a processor or programmable circuit to perform operations for identification of a vapor sample or chemicals in a vapor sample. The operations may include receiving a temperature profile defining a varying temperature level as a function of time or a set of discrete temperature levels, issuing a temperature control command in accordance with the temperature profile, the temperature control command for adjusting a temperature of one or more of a plurality of sensors included in a sensor array between at least two temperature levels, and identifying a vapor sample introduced to the sensor array or one or more chemicals in a vapor sample introduced to the sensor array based on a plurality of response patterns of the sensor array, each of the response patterns being a collection of responses of the plurality of sensors to the vapor sample with the one or more sensors being at a different temperature level from among the at least two temperature levels.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates a system for identification of a sample or chemicals in a sample according to an embodiment of the disclosure;

FIG. 2A is a graphical representation of a physical adsorption isobar;

FIG. 2B is a graphical representation of a chemical adsorption isobar;

FIG. 2C is a graphical representation of a comparison of combined physical and chemical adsorption isobars for two sensors having different sensory material coatings;

FIG. 2D is a graphical representation of sensor responses for two sensors having different sensory material coatings at two discrete temperatures;

FIG. 2E is a normalized version of the graphical representation of FIG. 2D;

FIG. 3 illustrates an example apparatus for identification of a sample or chemicals in a sample according to an embodiment of the disclosure;

FIG. 4A is a graphical representation of the responses of sixteen sensors at three different stabilized temperatures;

FIG. 4B is a graphical representation of the responses of sixteen sensors at three temperature levels as the temperature is ramped;

FIG. 4C is a graphical representation of temperature response profiles of four sensors;

FIG. 4D is a graphical representation of sensor responses of the four sensors at five discrete temperatures along the temperature response profiles;

FIG. 5A illustrates an example of the contents of a sensor response library of the apparatus;

FIG. 5B illustrates another example of the contents of the sensor response library of the apparatus;

FIG. 6 illustrates an alternative sensor array of the system;

FIG. 7 illustrates an alternative temperature controller configuration of the system;

FIG. 8 is an example operational flow according to an embodiment of the disclosure; and

FIGS. 9A and 9B illustrate an example of a computer in which the apparatus of FIG. 3, the operational flow of FIG. 8, and/or other embodiments of the disclosure may be wholly or partly embodied, with FIG. 9A illustrating the computer and FIG. 9B being a block diagram of a system unit of the computer.

### DETAILED DESCRIPTION

The present disclosure encompasses various embodiments of systems, methods, and apparatuses for identification of a sample or chemicals in a sample. The detailed description set forth below in connection with the appended drawings is intended as a description of several contemplated embodiments, and is not intended to represent the only form in which the disclosed invention may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

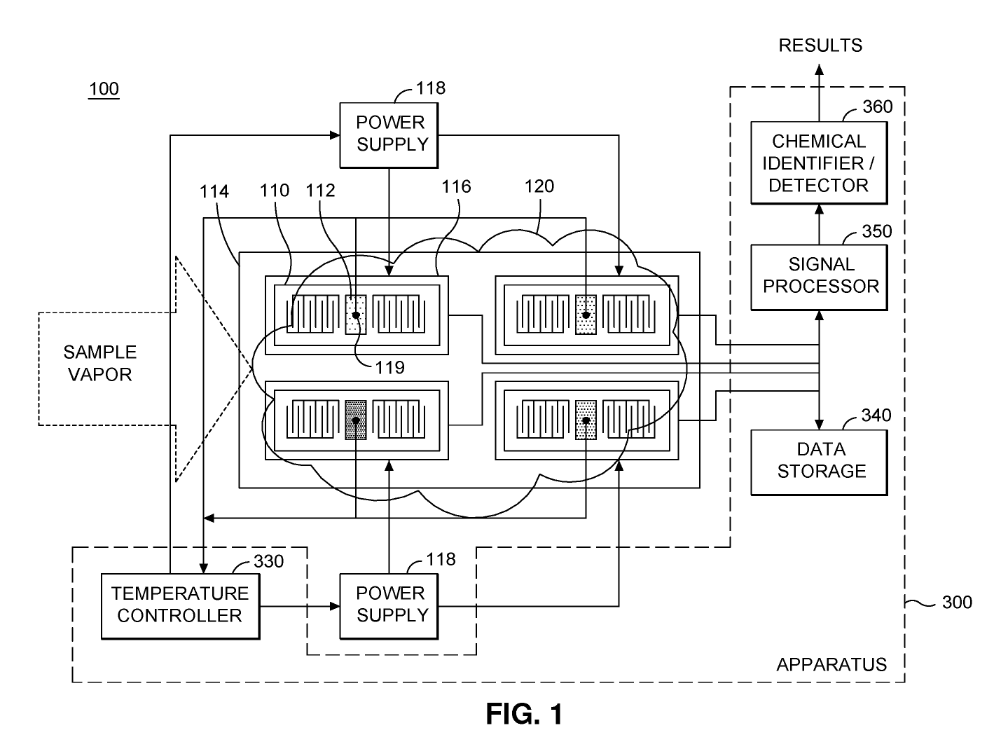

FIG. 1 illustrates a system 100 for identification of a sample or chemicals in a sample according to an embodiment of the disclosure. A sensor array including a plurality of sensors 110 coated with different sensory material coatings 112 is arranged on a sensor board 114. In the example shown in FIG. 1, each of the sensors 110 is a delay-line type surface acoustic wave (SAW) sensor, which detects an oscillation frequency change due to mass loading on the sensory material coating 112 coated on its surface. Depending on the adsorption affinity of the sensory material coating 112 (or in some cases lack of a sensory material coating 112), each of the sensors 110 may adsorb more or less of a given chemical or combination of chemicals, thus producing a different sensor response. Thus, when a vapor sample 120 (e.g. a substance to be classified/identified for purposes of disease detection or diagnosis) is introduced to the sensor array, a collection of responses of the plurality of sensors 110 produces a response pattern that may be indicative of a chemical or combination of chemicals in the vapor sample 120. Since the response pattern may depend on the temperature of each sensor 110, a temperature controller 330 may be provided to adjust the temperature of one or more of the sensors 110. A plurality of response patterns may thus be collected with each response pattern collected at a different temperature level. Based on the plurality of response patterns, a chemical identifier (i.e. detector) 360 may identify/classify the vapor sample 120 or identify the chemical or combination of chemicals in the vapor sample 120.

Since the sensor array of FIG. 1 may be used without first separating chemicals by gas chromatography, each of the sensors 110 may react to more than one chemical in a given vapor sample 120. Therefore, in general, the response Sₜⱼ of a given sensor j at a given temperature t may be represented by

Sₜⱼ=Σᵢ₌₁ⁿ(aₜⱼᵢcₜⱼᵢ) (Eq. 1)

where n is the number of chemicals that react to the sensor j or another sensor 110 in the sensor array, aₜⱼᵢ is the hypothetical response to chemical i that sensor j would exhibit at temperature t if chemical i were at 100% concentration in the vapor sample 120, and cₜⱼᵢ is a response coefficient for the given temperature t, sensor j, and chemical i based on the actual chemical makeup of the vapor sample 120. The response coefficient cₜⱼᵢ may be related to the concentration of chemical i in the vapor sample 120 and other factors, such as competition among the chemicals in the vapor sample 120. A response pattern of the sensor array can be represented by Sₜ₁, Sₜ₂, . . . , Sₜₘ for a sensor array of m sensors 110.

It has been found that the responses Sₜⱼ of individual sensors 110 can vary greatly depending on the temperature t, due to both the change in aₜⱼᵢ at different temperatures as the chemicals of the vapor sample 120 react differently with the sensory materials 112 and the change in cₜⱼᵢ at different temperatures as the chemicals of the vapor sample 120 react with each other. By establishing a response pattern Sₜ₁, Sₜ₂, . . . , Sₜₘ at each of a plurality of different temperatures t, the accuracy of identifying/classifying the vapor sample 120 or identifying the chemicals in the vapor sample 120 can be greatly improved as compared to using only a single response pattern Sₜ₁, Sₜ₂, . . . , Sₜₘ.

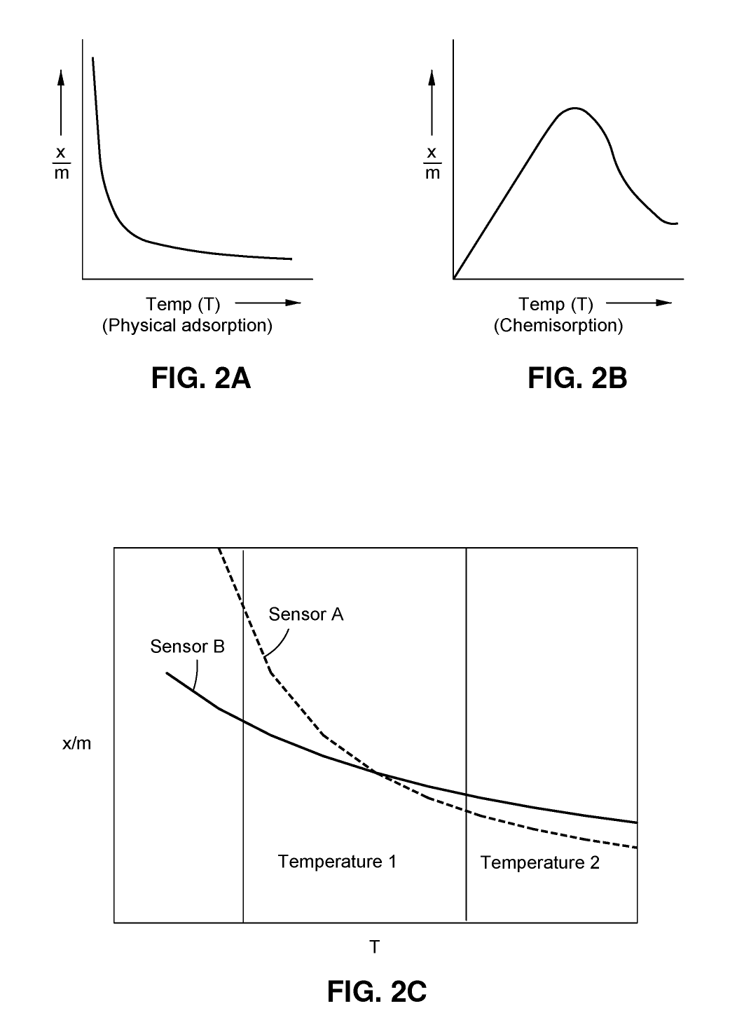

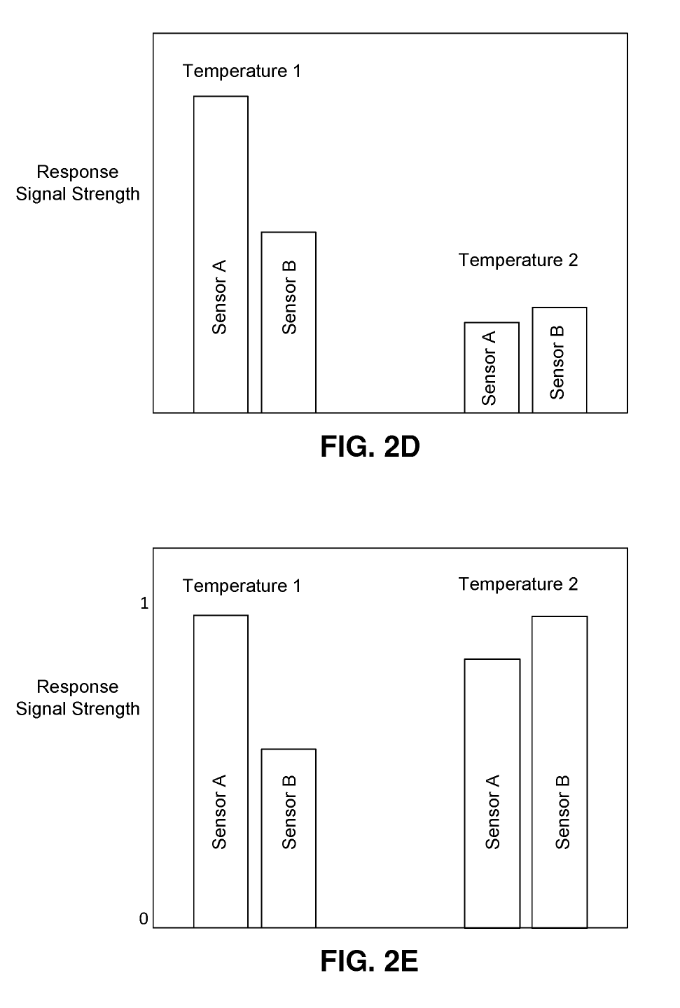

FIGS. 2A and 2B are graphical representations of physical and chemical adsorption isobars, respectively, with adsorption capability x/m (ratio of adsorbate mass x to adsorbent mass m) shown as a function of temperature T at constant pressure. The isobar of FIG. 2A represents a typical physical adsorption isobar, in which it can be observed that the adsorption capability x/m decreases with increased temperature T. The isobar of FIG. 2B represents a typical chemical adsorption (chemisorption) isobar, in which it can be observed that the adsorption capability x/m first increases with temperature T as adsorption sites are activated and then decreases at higher temperature T. In general, a sensor 110 may adsorb the chemicals of a vapor sample 120 by a combination of physical and chemical adsorption processes, resulting in an adsorption capability x/m having a complex temperature dependency that is different for each chemical of the vapor sample 120.

FIG. 2C is a graphical representation of a comparison of combined physical and chemical adsorption isobars for two sensors 110 having different sensory material coatings 112. In the example of FIG. 2C, the adsorption capability x/m for a given chemical is shown over a range of temperatures T including Temperature 1 and Temperature 2. At Temperature 1, the adsorption capability x/m of Sensor A is higher than the adsorption capability x/m of Sensor B, while at Temperature 2, the adsorption capability x/m of Sensor A is lower than the adsorption capability x/m of Sensor B. As can be seen, this relationship between the adsorption capabilities x/m of different sensors 110 may occur even where both adsorption capabilities x/m exhibit temperature dependence having the same sign (e.g. negative temperature dependence as shown in FIG. 2C).

FIG. 2D is a graphical representation of sensor responses at two discrete temperatures (Temperature 1 and Temperature 2) for two sensors 110 (Sensor A and Sensor B) having different sensory material coatings 112, and FIG. 2E is a normalized version of the graphical representation of FIG. 2D. As discussed above with respect to FIG. 2C, the relative adsorption capability between two sensors 110 may be different at different temperatures. As a result, as shown in FIG. 2D, it may often be the case that Sensor A exhibits a greater sensor response than Sensor B to the same chemical or to the same vapor sample 120 (e.g. due to a greater degree of adsorption of one or more chemicals) at a first temperature T1 while Sensor B exhibits a greater sensor response than Sensor A at a second temperature T2. The relative difference is emphasized in the normalized representation of FIG. 2E. The system 100 (see FIG. 1) may take advantage of these different sensor response patterns at different temperatures in order to identify/classify a vapor sample 120 or the chemicals(s) of a vapor sample 120 with more accuracy than can be achieved at an isothermal condition.

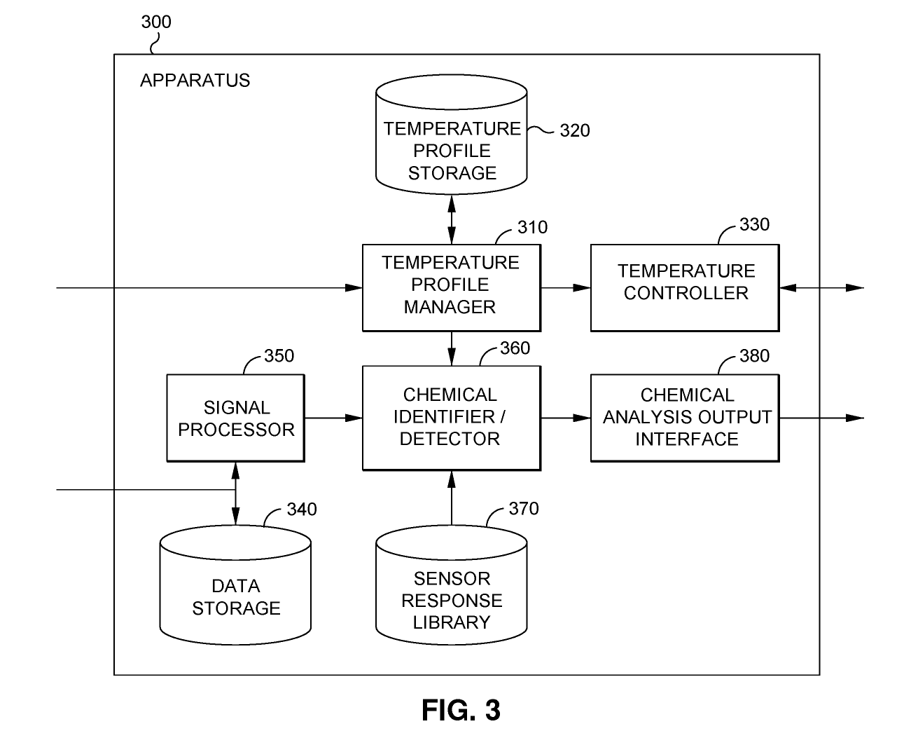

FIG. 3 illustrates an example apparatus 300 for identification of a sample or chemicals in a sample according to an embodiment of the disclosure. A simplified depiction of the apparatus 300 is shown in FIG. 1 in relation to the system 100. The apparatus 300 may adjust a temperature of one or more of the sensors 110 of the sensor array between at least two temperatures while the sensors 110 are exposed to a vapor sample 120 to be identified. The apparatus 300 may further receive the resulting sensor response data from the sensors 110 and identify/classify the vapor sample 120 or one or more chemicals in the vapor sample 120 based on response patterns of the sensor array at different temperatures. The apparatus 300 may include a temperature profile manager 310, a temperature profile storage 320, a temperature controller 330, a data storage 340, a signal processor 350, a chemical identifier (i.e. detector) 360, a sensor response library 370, and a chemical analysis output interface 380.

The temperature profile manager 310 may manage a temperature profile defining a varying temperature as a function of time or a set of discrete temperature levels. The temperature profile manager 310 may, for example, function as a temperature profile input interface for receiving the temperature profile from outside the apparatus 300 and storing the received temperature profile in the temperature profile storage 320 for use by the apparatus 300. The temperature profile manager 310 may, for example, receive the temperature profile from an external storage or from a computer or server through a wired or wireless network such as the Internet, WAN, and/or LAN. As another example, the temperature profile manager 310 may receive the temperature profile as a series of user input commands for creating a temperature profile from scratch, e.g. via any combination of input device(s) including, for example, mouse, keyboard, touchscreen, eye tracking, voice, and/or gestures. The temperature profile manager 310 may further function as a temperature profile editor for modifying an existing temperature profile stored in the temperature profile storage 320.

The temperature controller 330 may receive the temperature profile stored in the temperature profile storage 320 from the temperature profile manager 310. The temperature controller 330 may then instruct one or more heater/coolers 116 (see FIG. 1), e.g. thermoelectric coolers that can raise and lower temperature, to adjust the temperature of one or more of the sensors 110 while the sensors 110 are exposed to the vapor sample 120. The temperature controller 330 may, for example, issue a temperature control command to the heater/cooler(s) 116 to adjust the temperature of the sensor(s) 110 in accordance with the temperature profile. For example, the temperature profile may define a set point of the temperature controller 330, and the temperature controller 330 may issue temperature control commands to one or more power supplies 118 of the heater/cooler(s) 116 as a function of the set point defined by the temperature profile and a feedback signal received from one or more temperature sensors 119 (e.g. as a function of the difference between the set point and the feedback signal). As shown in FIG. 1, the temperature sensor(s) 119 may be disposed on a sensory area of the sensor(s) 110 (e.g. beneath the sensory material(s) 112). In this way, the temperature controller 330 may control the heater/cooler(s) 116 to maintain a temperature of the sensor(s) 110 corresponding to the temperature profile stored in the temperature profile storage 320.

The signal processor 350 may receive sensor response data generated by the sensors 110 of the sensor array, process the sensor response data, and store the processed sensor data in the data storage 340. Processing of sensor data by the signal processor 350 may include converting analog response data (e.g. oscillation frequency as a function of time in the case of a SAW sensor) to digital data at a sampling frequency (e.g. 50 Hz) or at a plurality of discrete instances, filtering the data, normalizing the data, Fourier transforming the data, and/or processing the data in any other way to make the sensor data usable as a measure of adsorption or other reaction affinity to the chemical(s) of the vapor sample 120. The processed data may be associated with a time stamp or sample number and stored in the data storage 340 in association therewith.

The chemical identifier 360 may identify the vapor sample 120 or a set of one or more chemicals in the vapor sample 120 based on the sensor data processed by the signal processor 350. For example, the chemical identifier 360 may identify the vapor sample 120 or chemical(s) based on a plurality of response patterns of the sensor array, each of the response patterns being a collection of responses of the plurality of sensors 110 to the vapor sample 120 taken at a different temperature level. As a specific example, the chemical identifier 360 may receive processed data of a single analysis run (to identify a single vapor sample 120) from the signal processor 350, where each of the data points is associated with a sensor ID and a time stamp or sample number. The chemical identifier 360 may further receive the temperature profile associated with the run from the temperature profile manager 310, with the temperature profile indicating an association between temperature levels and times or sample numbers. By matching the time stamps or sample numbers of the sensor data with the temperature profile, the chemical identifier 360 may associate each data point of the sensor data with the temperature of the sensor 110 at the time the sensor data was collected. In this way, the chemical identifier 360 may construct two or more response patterns corresponding to two or more temperatures t of the temperature profile, where each response pattern is a collection of all of the m sensor responses Sₜ₁, Sₜ₂, . . . , Sₜₘ at a specific temperature t. For example, in the case of two temperatures t=t₁ and t=t₂, the chemical identifier 360 may construct two response patterns Sₜ_{₁}₁, Sₜ_{₁}₂, . . . , Sₜ_{₁}ₘ and Sₜ_{₂}₁, Sₜ_{₂}₂, . . . , Sₜ_{₂}ₘ. The chemical identifier 360 may then compare the response patterns to a sensor response library 370 that includes a table of known response patterns at different temperatures. In this way, the chemical identifier 360 may search the sensor response library 370 for a match between each of a plurality of response patterns and one or more chemicals in the sensor response library 370.

In the above example, it is described that the chemical identifier 360 may associate each data point of the sensor data with a corresponding temperature of the sensor 110 by matching time stamps or sample numbers of the sensor data with the temperature profile. However, the disclosed embodiments are not intended to be limited to this particular methodology. For example, rather than matching sensor data to a temperature profile, the sensor data may be collected together with temperature data (i.e. the data may be “temperature stamped”). In this way, a measured temperature, rather than a target temperature, may be associated with each data point of sensor data. For example, the signal processor 350 may receive temperature data from temperature sensor(s) 119 in addition to receiving the raw analog sensor data of the sensors 110. The signal processor 350 may then sample both the temperature data and the sensor data according to the same sampling frequency and store each data point of sensor data in the data storage 340 in association with a corresponding measured temperature. In this case, since the incoming sensor data is already associated with the temperature of the sensor 110 at the time the sensor data was collected, the chemical identifier 360 may not need to associate each data point of the sensor data with a temperature using the temperature profile and may simply proceed with constructing a response pattern Sₜ₁, Sₜ₂, . . . , Sₜₘ at each temperature of interest and comparing the response patterns to the sensor response library 370.

The chemical analysis output interface 380 outputs one or more of various chemical analysis outputs of the apparatus 300 for use by a downstream device or user. For example, the outputs may be stored, uploaded to a server, printed, or otherwise made available for viewing or analysis. The various outputs of the apparatus 300 include, for example, singly or in combination, an identification/classification of the vapor sample 210 as determined by the chemical identifier 360, an identification of one or more chemicals present in the vapor sample 120 as determined by the chemical identifier 360, raw or processed sensor data and/or temperature data at any of various stages of processing by the signal processor 350, error reports related to failed attempts by the chemical identifier 360 to identify the vapor sample 120 or chemicals in the vapor sample 120, etc. Such outputs may also be displayed on a screen in relation to a user query as an intermediate step in a process performed by the apparatus 300.

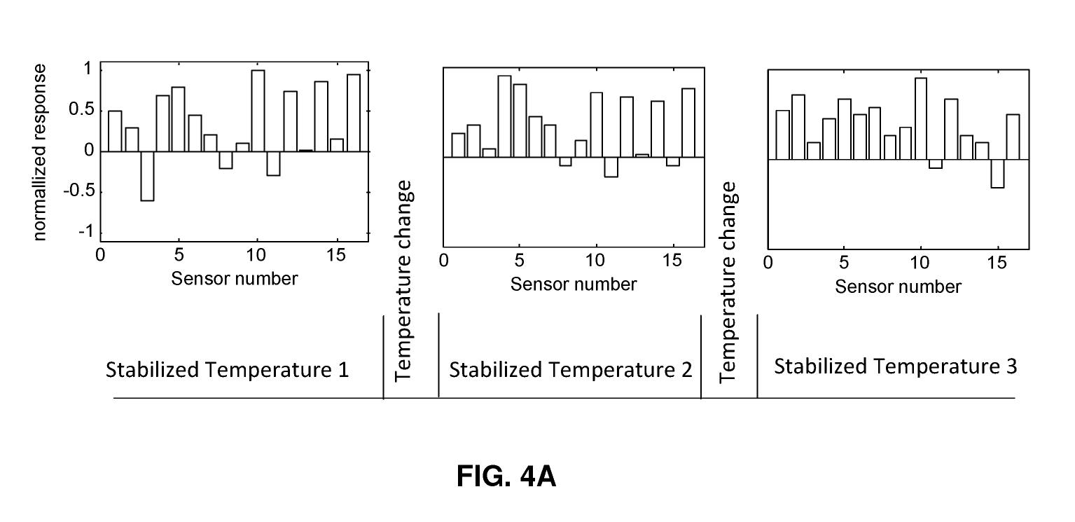

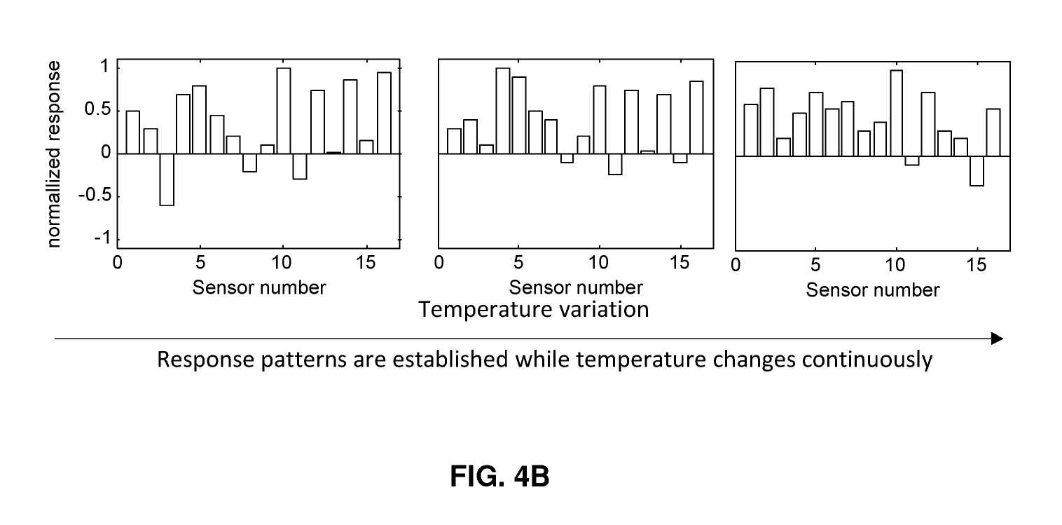

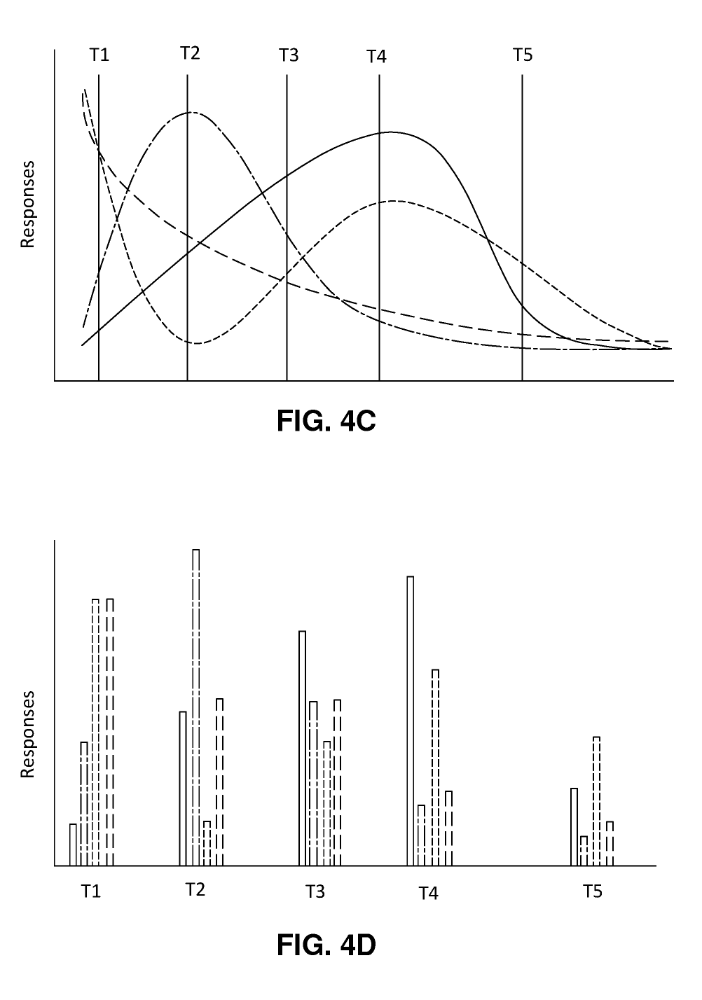

FIGS. 4A and 4B are graphical representations of the responses of a sensor array of sixteen sensors 110 at three different temperatures. As shown, the sixteen sensor responses making up each of the three response patterns may vary significantly at each of the three temperatures Temperature 1, Temperature 2, and Temperature 3. As contemplated by the disclosed embodiments, the system 100 may take advantage of this significant difference between the three response patterns by separately comparing such temperature-specific response patterns to temperature-specific library data, resulting in a highly accurate analysis.

In the example of FIG. 4A, the three temperatures Temperature 1, Temperature 2, and Temperature 3 are stabilized temperatures separated by periods of temperature change as shown, i.e. periods during which the temperature of the sensors 110 is adjusted. The three response patterns are constructed from sensor data that has stabilized after each temperature adjustment. This may be referred to as the stabilized temperature method. Because the adsorption or other reaction of the vapor sample 120 with the sensory material coating 112 may not be an instantaneous process, there may be a period of settling each time the temperature is adjusted during which the state of the adsorption or other reaction is not at equilibrium. Thus, in order for the response patterns to accurately reflect the reaction affinity of each sensor 110 at each temperature, there may be a waiting period to allow the sensor data to stabilize at each temperature. Such waiting period may be predetermined by incorporating waiting periods into a temperature profile. For example, the temperature profile may be a step-function where each temperature value is held for a waiting period that is expected to be long enough to allow the reactions between the sample vapor 120 and the sensors 110 to reach equilibrium. Alternatively, the waiting period may be determined during the analysis run as sensor data is collected. In this case, a temperature profile may only specify temperature values without any predetermined time dependence or sample number dependence, and the temperature of the sensors 110 may be adjusted to each specified value only after it is determined based on the sensor data that equilibrium has been reached (either automatically or by a person overseeing the run). The process can be repeated until a desired number of temperature levels is tested. In this latter case, where the temperature profile only specifies temperature values without any time dependence or sample number dependence, the response patterns may be established using “temperature stamped” sensor data as described above, rather than by matching the sensor data to the temperature profile used for the analysis run.

In the example of FIG. 4B, the three temperatures Temperature 1, Temperature 2, and Temperature 3 are three temperature levels along a temperature ramp. In this case, rather than wait for sensor response equilibrium at each temperature as in FIG. 4A, the response patterns are established while the temperature changes continuously throughout the run or throughout a portion of the run. This may be referred to as the dynamic temperature ramp method. For example, the temperature may be continuously ramped at one or more predetermined rates over a range of temperature levels including Temperature 1, Temperature 2, and Temperature 3. The one or more predetermined rates, as well as other parameters of the ramp (e.g. ramp direction(s), start and end points, etc.), may be completely specified by a temperature profile corresponding to the analysis run. Since the temperature changes continuously during the analysis run, sensor data may be collected without the reactions of the sensors 110 to the vapor sample 120 reaching equilibrium. However, the entire ramping process is repeatable according to the same temperature profile, and so the response patterns may nevertheless be reproducible and may be matched against known response patterns established under similar conditions.

FIG. 4C is a graphical representation of temperature response profiles of four sensors 110. A temperature response profile represents the output of a single sensor 110 over a range of temperatures. In the example of FIG. 4C, four temperature response profiles, corresponding to four sensors 110, are shown over a range of temperatures including temperatures T1, T2, T3, T4, and T5. In a case where the response of a sensor 110 to the vapor sample 120 quantifies a degree of adsorption of the vapor sample 120 to the sensor 110 (e.g. as in the case of the SAW sensors 110 of the present example), one or more temperature response profile like those shown in FIG. 4C may be generated so as to represent a desorption or adsorption profile of the sensor 110 or of the plurality of sensors 110. For example, during an analysis run (or likewise, during the collection of data of a known vapor sample 120 for purposes of training the sensor response library 370), the temperature of one or more sensors 110 may be initially held at a temperature level associated with a high degree of adsorption (e.g. a low temperature) until the response(s) of the one or more sensors 110 reach equilibrium. Subsequently, the temperature may be adjusted in a direction that reduces the degree of adsorption (e.g. the temperature may be ramped up). The resulting plurality of response patterns may then be arranged as a desorption profile beginning with a response pattern that is a collection of responses of the plurality of sensors 110 to the vapor sample 120 with the one or more sensors 110 being at the temperature level associated with the high degree of adsorption. Alternatively, the temperature of one or more sensors 110 may be initially held at a temperature level associated with a low degree of adsorption (e.g. a high temperature) until the response(s) of the one or more sensors 110 reach equilibrium. Subsequently, the temperature may be adjusted in a direction that increases the degree of adsorption (e.g. the temperature may be ramped down). The resulting plurality of response patterns may then be arranged as an adsorption profile beginning with a response pattern that is a collection of responses of the plurality of sensors 110 to the vapor sample 120 with the one or more sensors 110 being at the temperature level associated with the low degree of adsorption. It is noted that the plurality of response patterns may be termed “desorption profile” or “adsorption profile” for an entire sensor array even when the temperature is adjusted for only a portion of the sensors 110 in the array. In some cases, the terms “desorption profile” and “adsorption profile” may also be used to describe an individual temperature response profile (i.e. for an individual sensor 110), e.g. a single one of the four temperature response profiles shown in FIG. 4C, where the temperature profile of the sensor 110 has been generated by ramping the temperature beginning with a temperature level associated with a high or low degree of adsorption as described.

FIG. 4D is a graphical representation of sensor responses of the four sensors 110 at five discrete temperatures T1, T2, T3, T4, and T5 along the temperature response profiles shown in FIG. 4C. As such, each of the five sets of sensor responses (i.e. the four sensor responses at T1, the four sensor responses at T2, the four sensor responses at T3, the four sensor responses at T4, and the four sensor responses at T5) is a response pattern of the four sensors 110. The five response patterns collectively may constitute a “desorption profile” or “adsorption profile” of the sensor array in a case where the temperatures T1, T2, T3, T4, and T5 were ramped beginning with a high or low degree of adsorption of one or more of the sensors 110 as described above. As will be described below in more detail, the combination of five response patterns (which may constitute a “desorption profile” or “adsorption profile” of the sensor array) may itself also be regarded as a response pattern of the sensor array, with the response pattern representing the output of the plurality of sensors 110 over a single temperature profile (e.g. a temperature ramp) rather than at a single temperature (e.g. T1, T2, T3, T4, or T5).

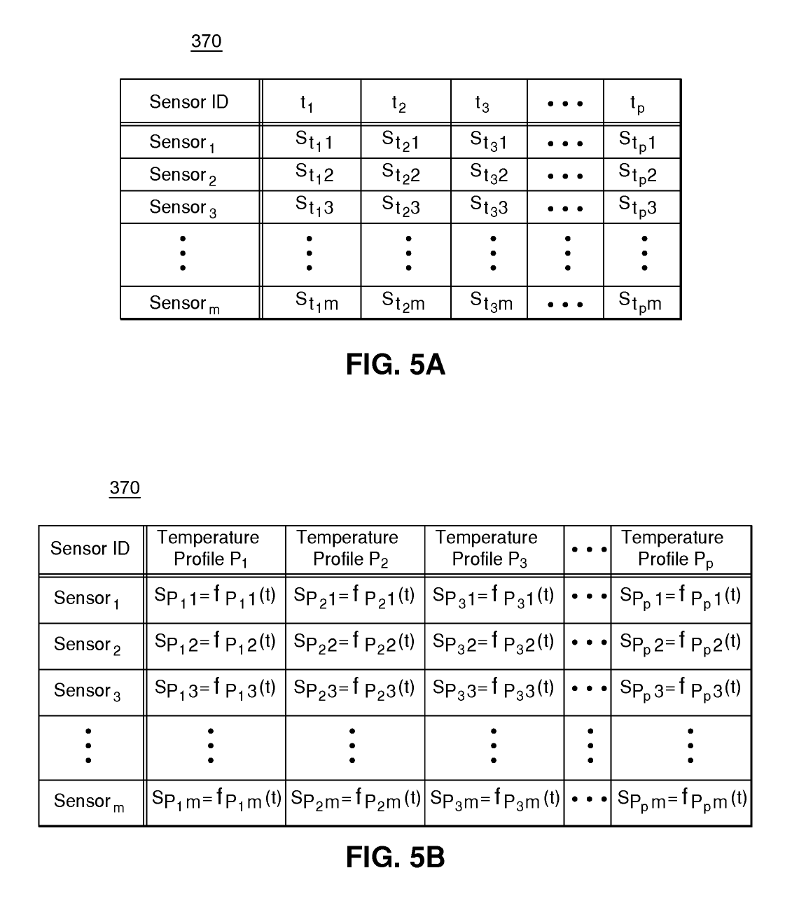

FIG. 5A illustrates an example of the contents of a sensor response library 370 of the apparatus 300. As describe above, the sensor response library 370 may include a table of known response patterns at different temperatures. When identifying a vapor sample 120 or chemicals in a vapor sample 120, the chemical identifier 360 may compare response patterns of the sensors 110 to the known response patterns of the sensor response library 370. As shown in FIG. 5A, the contents of the sensor response library 370 may be organized by Sensor ID and temperature for each of a plurality of known chemicals or combinations of chemicals (including, in some cases, known vapor samples or vapor sample classifications with the precise chemicals being unknown). That is, the entire table of FIG. 5A may be associated with a particular chemical or combination of chemicals. As such, the data in a column having a given header t represents a response pattern Sₜ₁, Sₜ₂, . . . , Sₜₘ that is expected to be observed when the array of sensors 110 having Sensor IDs Sensor₁ to Sensorₘ are exposed at temperature t to the chemical(s) associated with the table. For instance, if the sensors 110 are exposed to the chemical(s) associated with the table of FIG. 5A at temperature t₂, the expected response pattern would be Sₜ_{₂}₁, Sₜ_{₂}₂, . . . , Sₜ_{₂}ₘ as can be read from the column having the header t₂. The contents of the sensor response library 370 may be organized as a plurality of such tables, one for each chemical or combination of chemicals for which known response patterns are available. It is noted that in some cases it may be preferred for each such table to correspond to a combination of chemicals (e.g. an entire vapor sample or classification of a vapor sample) rather than to a single chemical, since competition between chemicals in a vapor sample 120 may influence the responses of the sensors 110.

When the chemical identifier 360 attempts to match data of an analysis run to the known response patterns stored in the sensor response library 370, a perfect match may not be possible. For example, due to measurement error, noise, trace contaminants, etc., it may be the case that some but not all of a response pattern matches the expected response pattern of a chemical or combination of chemicals in the sensor response library 370. In such cases, the closest match may be regarded as a positive identification. For instance, in an example where response patterns of sixteen sensors 110 are taken at three temperatures t₁, t₂, and t₃ (see, e.g., FIG. 4A), there may be only partial matching of the forty-eight (sixteen times three) data points to any particular chemical or combination of chemicals, but the partial matching may still be strongly indicative of one particular chemical or combination of chemicals. For example, the sensor responses of Sensor₁ through Sensor₁₄ at t₁, the sensor responses of Sensor₁ through Sensor₁₀ at t₂, and the sensor responses of Sensor₁ through Sensor₁₅ at t₃ may all match the expected sensor responses of the same chemical or combination of chemicals (e.g. Chemical A), with the remaining sensor responses of Sensor₁₅ and Sensor₁₆ at t₁, Sensor₁₁ through Sensor₁₆ at t₂, and Sensor₁₆ at t₃ matching expected sensor responses of various other chemicals in the sensor response library 370. In such a situation, it can be understood that Chemical A (i.e. the chemical or combination of chemicals that most closely matches the response patterns of the analysis run) may be regarded as a positive identification. It is also contemplated that the closest match above a predetermined matching threshold (e.g. 75% of data points) may be regarded as a positive identification, with an error (i.e. “no match”) being returned if no single chemical or combination of chemicals is matched by a number of data points exceeding the predetermined matching threshold.

The data stored in the sensor response library 370 may be established by analyzing known vapor samples 120 using the same or similar sensors 110 (e.g. sensors 110 having the same sensory material coatings 112), for example, by training the sensors 110 with known vapor samples 120 under specified conditions through machine learning, deep learning, or other artificial intelligence methods. For each known vapor sample 120, a response pattern exhibited by the sensors 110 at each of a plurality of temperatures t₁, t₂, t₃, . . . , tₚ can be stored as a table as shown in FIG. 5A and discussed above. By storing the individual components of each response pattern (i.e. the individual sensor responses Sₜ_{₁}₁, Sₜ_{₁}₂, etc.), matching can be done according to the closest match as described above even where entire response patterns do not match perfectly.

FIG. 5B illustrates another example of the contents of a sensor response library 370 of the apparatus 300. The example of FIG. 5B differs from the example of FIG. 5A in that each of the sensor responses S_{P}_{₁}₁, S_{P}_{₁}₂, etc. making up a given response pattern S_{P}_{₁}₁, S_{P}_{₁}₂, . . . , S_{P}_{₁}ₘ for the chemical or combination of chemicals associated with the table is itself not a single data point but a function of temperature (e.g. a curve). In other words, as shown in the table, each individual sensor response S_{P}_{₁}₁ may be notated as a function of temperature f(t), e.g. f_{P}_{₁}₁(t). Thus, each individual sensor response S_{P}_{₁}₁ may represent all of the expected data of a temperature response profile as shown in FIG. 4C, with a response pattern S_{P}_{₁}₁, S_{P}_{₁}₂, . . . , S_{P}_{₁}ₘ representing the output of the plurality of sensors 110 over a single temperature profile (e.g. a temperature ramp) rather than at a single temperature (e.g. T1, T2, T3, T4, or T5). A sensor response S_{P}_{₁}₁ may be established by analyzing known vapor samples 120 using the same or similar sensors 110 (e.g. sensors 110 having the same sensory material coatings 112) and fitting the resulting training data to a polynomial or other function. It is contemplated that matching the sensor data of an analysis run to such response patterns S_{P}_{₁}₁, S_{P}_{₁}₂, . . . , S_{P}_{₁}ₘ in the sensor response library 370 shown in FIG. 5B may be done by computing a distance between the data points of the analysis run and the functions stored in the sensor response library 370. The sensor response S_{P}_{₁}₁ closest to the data points may be regarded as a match, with the entire response pattern S_{P}_{₁}₁, S_{P}_{₁}₂, . . . , S_{P}_{₁}ₘ regarded as a positive identification if it is the response pattern that most closely matches the sensor data or if it is the closest match above a predetermined matching threshold (e.g. 75% of data points) as described above.

As shown in FIG. 5B, the sensor response library 370 may in some cases store multiple response patterns S_{P}_{₁}₁, S_{P}_{₁}₂, . . . , S_{P}_{₁}ₘ that differ in the temperature profile P₁, P₂, P₃, . . . Pₚ that was used to generate the training data. This may be useful in a case where the temperature profile of an analysis run is expected to influence the sensor data, such as in the example of FIG. 4B where the temperature changes continuously without waiting for the adsorption or other reactions to reach equilibrium. In such case, the shape (e.g. ramp direction, ramp rate, etc.) of the temperature profile may affect the sensor responses. Thus, it is contemplated that the sensor response library 370 may store separate data for each of various temperature profiles P₁, P₂, P₃, . . . Pₚ as shown. When matching to a sensor response library 370 that is organized in this way, the chemical identifier 360 may refer to the data in the column matching the temperature profile used for the analysis run. If multiple analysis runs are done using different temperature profiles, the chemical identifier 360 may refer to multiple columns when searching the sensor response library 370 for matches to further increase the accuracy of the analysis.

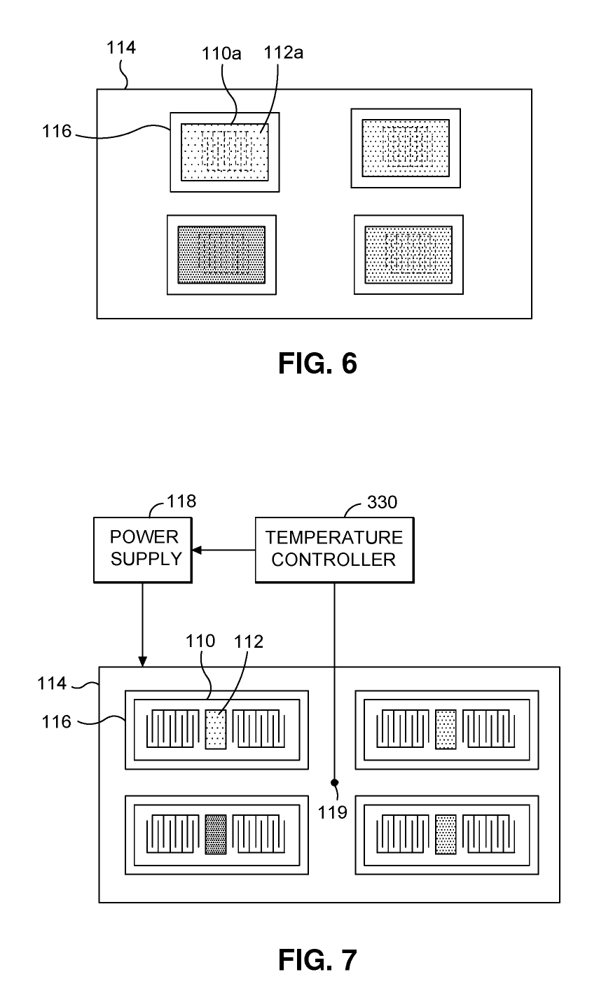

FIG. 6 illustrates an alternative sensor array of the system 100. The sensor array may be the same as that of FIG. 1 except that chemoresistant type sensors 110a having interdigital electrodes may be used instead of the SAW sensors 110 depicted in FIG. 1. The sensors 110a may be coated with sensory material coatings 112a that are the same as the sensory material coatings 112 shown in FIG. 1 except that the sensory material coatings 112a may be coated on the interdigital electrodes and not on the delay line as in the case of the SAW sensors 110. In general, any kinds of sensors can be used with the system 100 and apparatus 300, including, in addition to SAW sensors and chemoresistant type sensors, fluorescent sensors, metal oxide sensors or any other sensors that react to chemicals in the vapor sample 120. The disclosed embodiments are also not intended to be limited to using a sensor array of a single type of sensor 110, 110a. The sensor array may comprise a variety of different types of sensors 110, 110a, (e.g. two or more types, such as a few SAW sensors combined with chemoresistant type sensors), with each sensor 110 having the same or a different sensory material coating 112, 112a (or none at all), and the contents of the sensor response library 370 may be reflect the sensor array used. It is contemplated that sensory material coatings 112, 112a, as well as sensor types, may in some cases be carefully selected to respond to particular chemicals or combinations of chemicals when designing a suitable sensor array for a vapor sample 120. The number of sensors 110 can be four as shown in FIG. 1, two as shown in FIGS. 2C-2E, sixteen as shown in FIGS. 4A and 4B, or any other number.

FIG. 7 illustrates an alternative configuration of the system 100 with respect to the temperature controller 330. Unlike the example of FIG. 1, in which the temperature controller 330 may individually control the temperatures of one or more sensors 110 based on temperature measurements taken from temperature sensors 119 individually disposed with respect to each sensor 110 (e.g. on, near, or attached to each sensor 110), the configuration shown in FIG. 7 illustrates that a single temperature level may be set for the entire sensor array based on a single temperature sensor 119 disposed with respect to the sensor array, e.g. in the middle of the sensor board 114, under the assumption that the temperature of each sensor 110 is the same. Along the same lines, FIG. 7 schematically shows a single power supply line powering all four heater/coolers 116 via the sensor board 114. However, this is only one possible arrangement. For example, it is also contemplated that a single heater/cooler 116 may be provided extending underneath all of the sensors 110 of the sensor array. It should be noted that many temperature control arrangements are possible within the scope of the disclosed embodiments. In some cases (e.g. in the case of individual temperature control as shown in FIG. 1), it is envisioned that the temperatures of each sensor 110 may be adjusted separately, e.g. adjusted based on different temperature profiles, and/or that only some or one of the sensors 110 may have their temperature adjusted at all during the analysis run. Because the sensor response library 470 may store individual components of each response pattern (e.g. the individual sensor responses Sₜ_{₁}₁, Sₜ_{₁}₂, etc. in FIG. 5A or the individual sensor responses S_{P}_{₁}₁, S_{P}_{₁}₂, etc. in FIG. 5B), matching can be done even when each sensor 110 generates data at a different temperature.

The temperature control configuration of the system 100 may also depend on the type of heater/cooler(s) 116 used. As noted above, the heater/cooler(s) 116 may be thermoelectric coolers that may be powered by one or more power supplies 118. However, the heater/cooler(s) 116 may comprise radiation-based heating elements (e.g. infrared sources such as IR LEDs) that apply infrared radiation to heat the sensors 110. It is also contemplated that the heater/cooler(s) 116 may comprise heating wire connections that pass current (e.g. from one or more power supplies 118) through elements of the sensors 110 to directly heat the sensors 110 by resistive heating. In such cases, it is contemplated, for example, that heating and cooling commands may be applied by the temperature controller 330 to separate elements of the heating/cooling system.

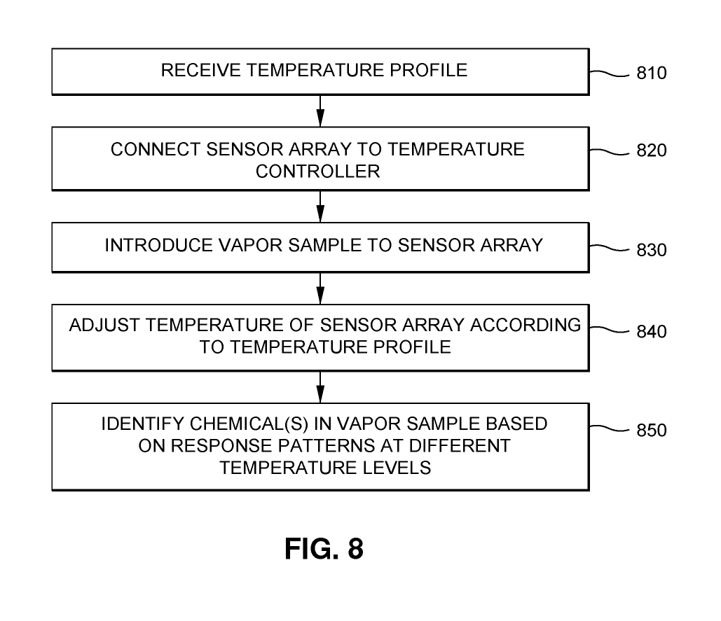

FIG. 8 is an example operational flow according to an embodiment of the disclosure. The operational flow of FIG. 8 will be described in relation to the embodiments of the system 100 of FIG. 1 and apparatus 300 of FIG. 3. However, the operational flow of FIG. 8 is not intended to be limited to these embodiments. First, a temperature profile is received in step 810. The temperature profile may define a varying temperature level as a function of time or may define only a set of discrete temperature levels. The temperature profile may be received by the temperature profile manager 310 or temperature controller 330 of the apparatus 300 shown in FIG. 3, e.g. by user input or by retrieval from the temperature profile storage 320. Alternatively, the temperature profile may be received (e.g. decided upon) by a person overseeing the analysis run, who will then use the temperature profile to manually adjust the temperature of the sensor array. Before, after, or simultaneously with step 810, the person overseeing the analysis run may connect the sensor array (e.g. sensors 110 shown in FIG. 1) to the temperature controller 330 in step 820. As noted above, there are many possible temperature control arrangements. For example, the temperature controller 330 (e.g. a computer system in which the temperature controller 330 is embodied) may be connected to one or more heater/cooler(s) 116 via one or more power supplies 118 of the heater/cooler(s) as shown in FIG. 1.

With the temperature profile having been received and the sensor array having been connected to the temperature controller 330, the operational flow of FIG. 8 may continue with introducing the vapor sample 120 to the sensor array in step 830. For example, the vapor sample 120 may be drawn to the sensor array using a vacuum pump or other mechanism (e.g. suction pump). The vapor sample 120 may originate as a vapor (e.g. a person's breath) or may originate as a solid or liquid that is vaporized prior to the analysis run. While the sensor array is exposed to the vapor sample 120, the operational flow may continue with step 840 of adjusting the temperature of one or more of the sensors 110 between at least two temperature levels (e.g. from a first temperature level to a second temperature level), as may be designated by the temperature profile. The temperature may be ramped up, ramped down, ramped down and up, stepped up and/or down, held for predetermined amounts of time or until sensor reactions reach equilibrium, or a combination thereof. Such adjustments may be done automatically by the temperature controller 330 or by manual operation of the temperature controller 330 by a person overseeing the analysis run. Adjustment by the temperature controller 330 may include the issuance of a temperature control command for adjusting the temperature of the one or more sensors 110.

At the completion of the analysis run (or simultaneous with the analysis run as data becomes available), in step 850, the vapor sample 120 or chemical(s) in the vapor sample 120 may be identified/classified based on response patterns at the different temperature levels of the analysis run. For example, the chemical identifier 360 or a person overseeing the analysis run may compare the sensor data (e.g. processed by the signal processor 350 and/or stored in the data storage 340) with known data stored in a sensor response library 370 as described above. In some cases (e.g. where the sensor data is stored in association with a time or sample number but not a temperature), further reference may be made to the temperature profile associated with the analysis run in order to match response patterns with temperatures as described above. By identifying/classifying the vapor sample 120 using not just a single response pattern but a plurality of response patterns, where each response pattern is a collection of responses of the sensors 110 at a different temperature level, the accuracy of identifying/classifying the vapor sample 120 can be greatly improved and a greater certainty can be established with respect to the results. Between analysis runs or after a series of analysis runs, the sensors 110 may be heated up to release any residue on the sensor 110 in preparation for the next vapor sample 120.

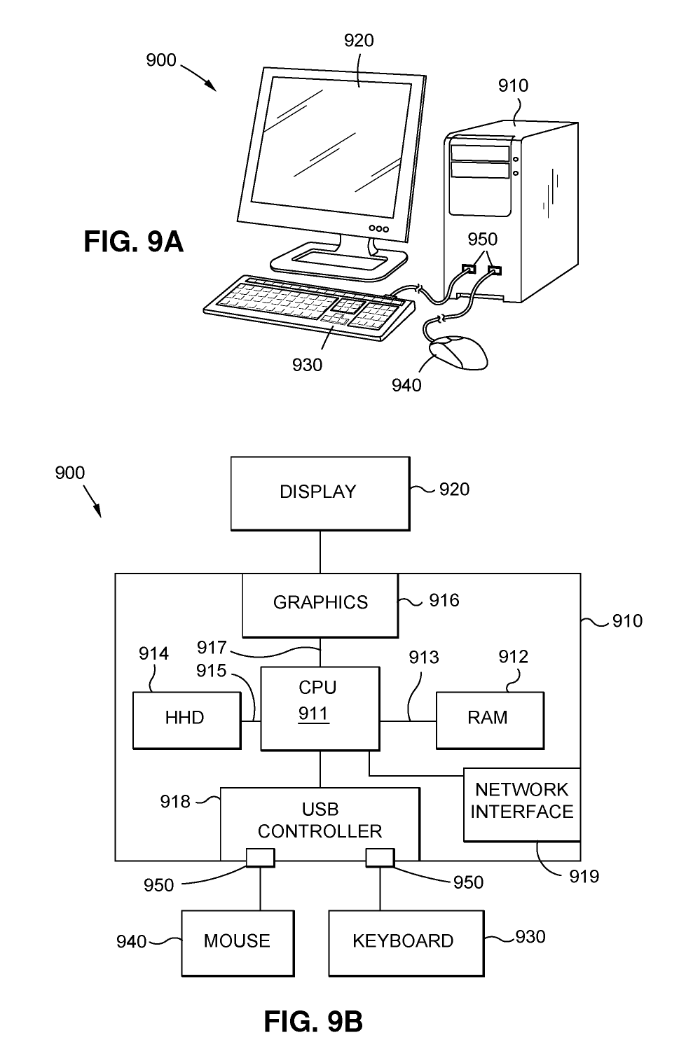

FIGS. 9A and 9B illustrate an example of a computer in which the apparatus of FIG. 3, the operational flow of FIG. 8, and/or other embodiments of the disclosure may be wholly or partly embodied, with FIG. 9A illustrating the computer and FIG. 9B being a block diagram of a system unit of the computer. The computer 900 according to the present embodiment, as shown in FIG. 9A, generally may include a system unit 910 and a display device 920. The display device 920 may produce a graphical output from the data processing operations performed by the system unit 910. Input devices including a keyboard 930 and a mouse 940, for example, may be manipulated by a user to generate corresponding inputs to the data processing operations, and may be connected to the system unit 910 via ports 950. Various other input and output devices may be connected to the system unit 910, and different interconnection modalities are known in the art.

As shown in the block diagram of FIG. 9B, the system unit 910 may include a processor (CPU) 911, which may be any conventional type. A system memory (RAM) 912 may temporarily store results of the data processing operations performed by the CPU 911, and may be interconnected thereto via a dedicated memory channel 913. The system unit 910 may also include permanent storage devices such as a hard drive 914, which may be in communication with the CPU 911 over an input/output (I/O) bus 915. A dedicated graphics module 916 may be connected to the CPU 911 via a video bus 9617, and may transmit signals representative of display data to the display device 920. As indicated above, the keyboard 930 and the mouse 940 may be connected to the system unit 910 over the ports 950. In embodiments where the ports 950 are Universal Serial Bus (USB) type, there may be a USB controller 918 that translates data and instructions to and from the CPU 911 for the external peripherals connected via the ports 950 or wirelessly connected such as via Bluetooth connectivity. Additional devices such as printers, microphones, speakers, and the like may be connected to the system unit 910 thereby.

The system unit 910 may utilize any operating system having a graphical user interface (GUI), such as WINDOWS from Microsoft Corporation of Redmond, Wash., MAC OS from Apple, Inc. of Cupertino, Calif., various versions of UNIX with the X-Windows windowing system, and so forth. The system unit 910 may execute one or more computer programs, with the results thereof being displayed on the display device 920. Generally, the operating system and the computer programs may be tangibly embodied in a computer-readable medium, e.g., the hard drive 914. Both the operating system and the computer programs may be loaded from the aforementioned data storage devices into the RAM 912 for execution by the CPU 911. The computer programs may comprise instructions, which, when read and executed by the CPU 911, cause the same to perform or execute the steps or features of the various embodiments set forth in the present disclosure.

For example, a program that is installed in the computer 900 can cause the computer 900 to function as an apparatus such as the apparatus 300 of FIG. 3. Such a program may act on the CPU 911 to cause the computer 900 to function as some or all of the sections, components, elements, databases, storages, libraries, engines, interfaces, managers, controllers, processors, identifiers, detectors, etc. of the apparatus 300 of FIG. 3 (e.g., the temperature profile manager 310, the chemical identifier 360, etc.). A program that is installed in the computer 900 can also cause the computer 900 to perform an operational flow such as that illustrated in FIG. 8 or a portion thereof. Such a program may, for example, act on the CPU 911 to cause the computer 900 to perform one or more of the steps of FIG. 8 (e.g., receive temperature profile 810, adjust temperature of sensor array according to temperature profile 840, identify chemical(s) in vapor sample based on response patterns at different temperature levels 850, etc.).

The above-mentioned program may be provided to the hard drive 914 by or otherwise reside on an external storage medium such as a DVD-ROM, optical recording media such as a Blu-ray Disk or a CD, magneto-optic recording medium such as an MO, a tape medium, a semiconductor memory such as an IC card, a mechanically encoded medium such as a punch card, etc. Additionally, program storage media can include a hard disk or RAM in a server system connected to a communication network such as a dedicated network or the Internet, such that the program may be provided to the computer 900 via the network. Program storage media may, in some embodiments, be non-transitory, thus excluding transitory signals per se, such as radio waves or other electromagnetic waves.

Instructions stored on a program storage medium may include, in addition to code executable by a processor, state information for execution by programmable circuitry such as a field-programmable gate arrays (FPGA) or programmable logic array (PLA).

Although certain features of the present disclosure are described in relation to a computer 900 with input and output capabilities including a keyboard 930 and mouse 940, specifics thereof are presented by way of example only and not of limitation. Any alternative graphical user interfaces such as touch interfaces and pen/digitizer interfaces may be substituted. The analogues of those features will be readily appreciated, along with suitable modifications to accommodate these alternative interfaces while still achieving the same functionalities.

Along these lines, the foregoing computer 900 represents only one exemplary apparatus of many otherwise suitable for implementing aspects of the present disclosure, and only the most basic of the components thereof have been described. It is to be understood that the computer 900 may include additional components not described herein, and may have different configurations and architectures. Any such alternative is deemed to be within the scope of the present disclosure.

Throughout the above disclosure, various methodologies are described for conducting an analysis run and identifying/classifying a vapor sample 120, including a stabilized temperature method as described in relation to FIG. 4A, a dynamic temperature ramp method as described in relation to FIG. 4B, collection of temperature response profiles as described in relation to FIGS. 4C and 4D, matching of response patterns associated with individual temperature levels, matching of response patterns associated with temperature profiles (e.g. establishing desorption/adsorption profiles), matching to individual data points as described in relation to FIG. 5A, matching to curves as described in relation to FIG. 5B, etc. It is also contemplated that a combination of such methodologies can be used. For example, response patterns may be established for a certain temperature range dynamically, while also being established at stabilized temperatures. As another example, a vapor sample 120 may be identified/classified using a combination of selected response profiles over specified detection times or within specified temperature ranges (e.g. desorption/adsorption profiles) as described in relation to FIG. 5B in addition to selected response profiles at a few individual temperature levels as described in relation to FIG. 5A. The combination of methodologies used may be optimized depending on the application.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method for identification of a vapor sample or one or more chemicals in a vapor sample, the method comprising:
introducing a vapor sample to a sensor array including a plurality of sensors;
adjusting a temperature of one or more of the plurality of sensors between at least two temperature levels; and
identifying the vapor sample or one or more chemicals in the vapor sample based on a plurality of response patterns of the sensor array, each of the response patterns being a collection of responses of the plurality of sensors to the vapor sample with the one or more sensors being at a different temperature level from among the at least two temperature levels.

2. The method of claim 1, wherein said adjusting includes continuously ramping the temperature at one or more predetermined rates over a range of temperature levels including the at least two temperature levels.

3. The method of claim 2, further comprising:
receiving a temperature profile defining a varying temperature level as a function of time,
wherein said continuously ramping the temperature is performed according to the temperature profile.

4. The method of claim 1, wherein said adjusting includes holding the temperature at each of the at least two temperature levels until the responses of the one or more sensors at that temperature level reach equilibrium.

5. The method of claim 4, further comprising:
receiving a temperature profile defining a set of discrete temperature levels,
wherein said holding the temperature at each of the at least two temperature levels is performed according to the temperature profile.

6. The method of claim 1, wherein the response of each of the plurality of sensors to the vapor sample quantifies a degree of adsorption of the vapor sample to the sensor.

7. The method of claim 6, wherein
said adjusting includes initially holding the temperature at a temperature level associated with a high degree of adsorption until the responses of the one or more sensors at that temperature level reach equilibrium and subsequently adjusting the temperature in a direction that reduces the degree of adsorption, and
the plurality of response patterns is arranged as a desorption profile beginning with a response pattern that is a collection of responses of the plurality of sensors to the vapor sample with the one or more sensors being at the temperature level associated with the high degree of adsorption.

8. The method of claim 6, wherein
said adjusting includes initially holding the temperature at a temperature level associated with a low degree of adsorption until the responses of the one or more sensors at that temperature level reach equilibrium and subsequently adjusting the temperature in a direction that increases the degree of adsorption, and
the plurality of response patterns is arranged as an adsorption profile beginning with a response pattern that is a collection of responses of the plurality of sensors to the vapor sample with the one or more sensors being at the temperature level associated with the low degree of adsorption.

9. The method of claim 1, wherein said identifying includes searching a sensor response library for a match between each of the plurality of response patterns and one or more chemicals in the sensor response library.

10. The method of claim 9, wherein the sensor response library stores known response patterns in association with chemicals or combinations of chemicals.

11. The method of claim 10, wherein individual components of the known response patterns are stored in the sensor response library in association with individual sensors.

12. The method of claim 10, wherein the known response patterns are stored in the sensor response library in association with the plurality of sensors of the sensor array.

13. The method of claim 12, wherein individual components of the known response patterns are stored in the sensor response library in association with individual sensors from among the plurality of sensors of the sensor array.

14. The method of claim 10, wherein the known response patterns are stored in the sensor library in association with temperature levels at which the known response patterns were determined.

15. The method of claim 15, wherein the known response patterns are stored in the sensor library in association with temperature profiles specifying how temperature was controlled during the determination of the known response patterns, each of the temperature profiles defining a varying temperature level as a function of time or a set of discrete temperature levels.

16. The method of claim 1, wherein each of the plurality of sensors is of a type selected from the group consisting of: surface acoustic wave (SAW), chemoresistant, fluorescent, and metal oxide.

17. The method of claim 1, wherein the plurality of sensors includes sensors of two or more types selected from the group consisting of: surface acoustic wave (SAW), chemoresistant, fluorescent, and metal oxide.

18. The method of claim 1, wherein at least two of the plurality of sensors are coated with different sensory material coatings that produce different sensor responses to the vapor sample.

19. A system for identification of a vapor sample or chemicals in a vapor sample, the system comprising:
a sensor array including a plurality of sensors;
a temperature controller that adjusts a temperature of one or more of the plurality of sensors between at least two temperature levels; and
a chemical identifier that identifies a vapor sample introduced to the sensor array or one or more chemicals in a vapor sample introduced to the sensor array based on a plurality of response patterns of the sensor array, each of the response patterns being a collection of responses of the plurality of sensors to the vapor sample with the one or more sensors being at a different temperature level from among the at least two temperature levels.

20. A non-transitory program storage medium on which are stored instructions executable by a processor or programmable circuit to perform operations for identification of a vapor sample or chemicals in a vapor sample, the operations comprising:
receiving a temperature profile defining a varying temperature level as a function of time or a set of discrete temperature levels;
issuing a temperature control command in accordance with the temperature profile, the temperature control command for adjusting a temperature of one or more of a plurality of sensors included in a sensor array between at least two temperature levels; and
identifying a vapor sample introduced to the sensor array or one or more chemicals in a vapor sample introduced to the sensor array based on a plurality of response patterns of the sensor array, each of the response patterns being a collection of responses of the plurality of sensors to the vapor sample with the one or more sensors being at a different temperature level from among the at least two temperature levels.
